(19) United States
(12) Patent Application Publication
(10) Pub. No.: US 2023/0000686 A1
(43) Pub. Date: Jan. 5, 2023

(54) COMPRESSION BANDAGE

(71) Applicant: KOB GMBH, Wolfstein (DE)

(72) Inventor: Ferdinand TAMOUÉ, Ludwigshafen am Rhein (DE)

(21) Appl. No.: 17/781,484

(22) PCT Filed: Dec. 14, 2020
(86) PCT No.: PCT/EP2020/085886

§ 371 Date: Jun. 1, 2022

(30) Foreign Application Priority Data
Dec. 17, 2019 (DE) 10 2019 134 780.7

(51) Int. Cl. A61F 13/00

(52) U.S. Cl. CPC A61F 13/00017; A61F 13/00038; A61F 13/00029; A61F 13/00042

(57) ABSTRACT

The invention relates to a compression bandage comprising a first non-woven laser (1) and at least one second non-woven layer (2) connected in a planar manner to the first non-woven layer (1), wherein superabsorbent fibers are provided in the first non-woven laser.

The invention relates to a compression bandage comprising at least two nonwoven layers.

Compression bandages are used in the prior art, for diabetic ulcers, for example. A problem here is that the clinical picture dictates that there are variety of issues that need to be borne in mind, these being on the one hand, sufficient compression, but also, on the other, a cushioning effect with respect to the limb that is to be treated.

One possibility is, for example, to apply a cushion and to apply a compression bandage over it.

In this connection there is a fundamental distinction to be made in compression therapy between the so-called working pressure and the so-called resting pressure, with the resting pressure being the pressure which is exerted by the compression means, in this case the compression bandage, on the limb when the limb is horizontal. The working pressure, then, is the pressure which is exerted on the limb when the muscles are moving. The working pressure is preferably to be 20 to 40 mm Hg above the resting pressure.

It is likewise known in this connection to use different types of bandage. Hence there are so-called long-stretch bandages known that exhibit a very high elasticity and frequently possess a stretching capacity of more than 200%, whereas short-stretch bandages are known which possess only low stretchability and only a low restoring force, but at a very early point do not allow any further stretching and so have the capacity to develop a comparatively high working pressure. Short-stretch bandages, conversely, develop only a low resistance over a comparatively long range, before then exerting a very high degree of limitation on the stretchability. The traditional short-stretch bandage compression therapy is a measure for treating venous disorders. In this case the materials for short-stretch bandages are manufactured generally from nonelastic materials and are elastified by finishing processes. However, there is a significant reduction in the elasticity during the treatment. This may lead to a reduction in the compressive pressure during use.

Known compression bandages are described in EP 2 275 062 A2, for example, which describes an inner, skin-facing elastic bandage having a stretched elastic substrate and a stretched foam layer, disposed on the skin-facing side of the substrate, and also a further stretched, self-adhesive elastic bandage, which is applied over the inner bandage.

Additionally known, from WO 2017/109209, for example, are compression bandages composed of a plurality of nonwoven plies connected to one another by stitching; these may optionally be combined with a further bandage plie.

To achieve high therapeutic reliability, it is desirable for the therapeutically required compressive pressure to be attained as effectively as possible. For this purpose there are a multiplicity of possibilities known in the prior art. For example, markers are known which deform on excessive stretching, allowing the therapist to recognize an excessive stretch and hence an excessive application pressure. A disadvantage here, however, is that trained personnel are always needed.

Furthermore, in the case of the treatment of oedemas or chronic wounds, the objective of reducing the existing oedemas and of enabling the accelerated transport of exudate away from the wound is often accompanied by the disadvantage that the wound exudate arising acts counter to the course of healing of the wound, particularly in the case of chronic wounds where such compression therapies are employed. The wound exudate thus contains, for example, enzymes which prevent the construction of an extracellular matrix. MMPs (matrix metalloproteinases) are examples of such enzymes. It is therefore necessary to apply additional wound contact layers beneath the compression bandage. In this case there is a risk that the wound contact layer will slip relative to the compression bandage and therefore not develop its effect as intended.

It is therefore desirable to provide a compression bandage—which may also be referred to alternatively as a compression dressing—which exhibits a high reliability in application and which at the same time exhibits good therapeutic properties.

In so far as the terms “layer” or “ply” are used, they refer to the same subject matter.

Proceeding from this prior art, the invention achieves the object by means of a compression bandage having the features of claim 1, with the first nonwoven layer consisting of a nonwoven material which includes superabsorbent fibers (SAF). A bandage of this kind has the advantage that the first nonwoven layer, as well as the fact that it can be worn in particular for several days without any finding of a marked drop in the compressive pressure, is additionally able to absorb and to bind wound fluids. It has emerged that superabsorbent fibers are able both to inhibit proteases via diffusive mechanisms and to compartmentalize them by direct binding, and so to remove them from the wound exudate and/or the wound. It has emerged more particularly that superabsorbent fibers are suitable for inhibiting proteases in chronic wounds. It has emerged, moreover, that metalloproteases are bound or compartmentalized by superabsorbent fibers, allowing these metalloproteases to be removed with the superabsorbent fibers from a wound fluid or a wound. Accordingly, by means of the superabsorbent fibers, it is possible to capture an excess of metalloproteases in chronic wounds in such a way that a natural course of healing can take place.

The first nonwoven layer here may consist of a nonwoven material into or to which the superabsorbent fibers are introduced or applied. According to one particular embodiment, the nonwoven material may also consist completely of superabsorbent fibers.

The second nonwoven layer and the first nonwoven layer are preferably connected to one another by means of stitchbonding methods via an elastic thread. The stitch length more particularly is 1.5 to 3 mm/r at a stitching thread tension of at most 4 cN. Connection is accomplished in particular in the unstretched state.

This design allows the at least two layers to be connected by means of the stitchbonding method, and in this case preferably by a Malimo or Maliwatt method, with the layers being connected preferably in the unstretched state by means of the elastic stitching thread.

The advantage with a stitchbonding method is that a connection may be performed simultaneously at two or more points and the two layers, after having been connected, can no longer be separated from one another. Via the selection of the stitch length in the longitudinal direction of the fabric from which the compression bandages are then made up, with stitch length referring to the distance between two stitches in the longitudinal stitching direction, and via the stitching thread tension, it is possible to adjust the stretchability of the elastic assembly, with the resultant elastic assembly composed of the two layers that is no longer separable by hand and yet is controllable. Depending on the selection of these parameters, the completed fabric contracts when tension is released, and creases are formed in the material.

The stitching technique and the stitch length of the stitching thread here are preferably regulated such that the fibers on the first nonwoven layer, on one side of the assembly composed of the two layers, possess skin comfort and equalization functions, and hence, ultimately, the compression bandage has in particular two discernibly different sides, which are highly functional for pressure equalization. Furthermore, the stitch length here must be adjusted in particular such that the desired absorbing and also skin-friendly properties of the skin-facing first nonwoven layer are maintained.

However, there are also other conceivable types of connection, such as, for example, by way of needling, adhesive connection, thermal connection, via weld points and/or via pressure. The connection here may be areal, including partially, or may be linear or pointwise.

The first nonwoven layer here is preferably the limb-facing side of a compression bandage, and the second nonwoven layer is preferably the second side that is applied to the first layer. Also conceivable, however, is an opposite arrangement, where the second nonwoven layer or any further layers provided allow the passage of exudate. In between them, i.e., between first and second nonwoven layers, or on the side of the second nonwoven layer that faces away from the first nonwoven layer, there may be further layers provided, with cushioning effect or with stabilizing or adhering functions. As well as further nonwoven layers, there may also be film layers or layers formed of elastic threads. These layers may be continuous or discontinuous and more particularly may be only partial or cover the full area of the first and/or second nonwoven layers.

Where a stitchbonding method is envisaged, the method used for connecting the layers is more particularly the Malimo or Maliwatt method, as known in the prior art. Accordingly the elasticity may have been obtained, for example, by overstitching a rigid nonwoven fabric or woven fabric with permanently elastic elastane threads in the longitudinal direction, employing the MALIWATT or Malimo stitchbonding technique. The MALIWATT or Malimo stitchbonding technique is described in Malimo Nahwirktechnologie [Malimo stitchbonding technology], Ploch, Böttcher, Scharch, VEB Fachbuchverlag Leipzig, 1978, 1^{st} edition.

One or both layers, i.e., the first nonwoven layer and the second nonwoven layer, may preferably be nonelastic, and are elastified only by the stitchbonding method, for example. This is the case preferably for further, optional layers as well. The elastification may alternatively be accomplished as well by the introduction or application of elastic threads or elastic films.

With further preference the first and second nonwoven layers are the same in terms of their extents. More particularly they are flush at the edges in the width direction. The two nonwoven layers lie areally one on the other.

According to one exemplary embodiment, the first nonwoven layer may be a nonwoven wadding layer, more particularly a thermofused nonwoven layer, which may optionally also have already undergone preliminary needling. In this case, with both methods, namely thermobonding and thermofusion, the fibers of the nonwovens are laid in a defined direction in a combing process and prepared in the form of nonwoven rolls in a textile functionalization process, and stabilized for further processing by means of temperature or by means of temperature and pressure. During the thermofusion method, fibers with different melting points are fused to one another by hot air dryers. The superabsorbent fibers as well are processed in this case.

In the thermobonding method, the fibers are fused by means of heat pressure between heated calender rolls.

The result in both cases are soft, homogeneous nonwoven fabrics which are suitable ideally and for technical applications.

The thermofusion method is more suitable for first nonwoven layers, owing to the absence of pressure, where this layer is to exhibit a cushioning function.

The second nonwoven layer may preferably be a thermobonded nonwoven. This thermobonded nonwoven preferably has only a low stretching capacity in combination with desired stiffness. The base material of the nonwoven employed for the first and/or second nonwoven layer is preferably polyester. Alternative materials suitable include viscose, cellulose, alginate, acetate, polyamide, polyethylene, polypropylene, or combinations of these. In principle the second nonwoven layer may likewise comprise superabsorbent fibers.

The superabsorbent fibers are preferably fibers composed of an organic material, which may comprise synthetic and/or natural material, such as, for example, agar, pectin and guar gum, or else synthetic materials, such as, for example, synthetic hydrogel polymers. Synthetic hydrogel polymers comprise, for example, carboxymethylcellulose, alkali metal salts of polyacrylic acids, alkali metal salts of polymethacrylic acids, polyacrylamides, polyvinyl alcohol, ethylene-maleic anhydride copolymers, polyvinyl ethers, hydroxypropylcellulose, polymers and copolymers of vinylsulfonic acid, polyacrylates, polymethacrylates, polyacrylate-polymethacrylate copolymers, polyacrylamides, and the like. Especially preferred as superabsorbent fibers are those fibers which are made of alkali metal salts of polyacrylic acids, alkali metal salts of polymethacrylic acids, polyacrylates, polymethacrylates or polyacrylate-polymethacrylate copolymers. The hydrogel polymers are preferably slightly crosslinked, in order to render the materials substantially insoluble in water. The crosslinking may be accomplished, for example, by irradiation or by the forming of covalent bonds, ionic bonds, van der Waals bonds or hydrogen bonds. Suitable materials are available from a variety of manufacturers, such as BASF and Stockhausen Inc.

Provision is made in particular here for the second absorbing layer to comprise, as absorbing material, a fibrous nonwoven which comprises a fiber mixture of superabsorbent fibers and supporting fibers. In this case, however, provision may also be made for the fibrous nonwoven to comprise superabsorbent fibers and supporting fibers and for the supporting fibers to be selected from the group composed of thermoplastic fibers and/or cellulose fibers and/or viscose fibers.

With very particular preference, the fiber mixture comprises superabsorbent fibers, viscose fibers, and thermoplastic fibers, where thermoplastic fibers employable are additionally, preferably, polypropylene or polyethylene fibers. These thermoplastic fibers may also take the form of what are called bicomponent fibers in the form of core-sheath fibers.

The second absorbing material here preferably comprises a fiber mixture which comprises more than 20 wt %, more particularly more than 30 wt %, preferably more than 40 wt %, preferably more than 50 wt %, and more preferably more than 60 wt % of superabsorbent fibers. This enables the provision of an absorbing cushion which exhibits particularly high free absorption. A wound contact layer furnished accordingly may remain on a wound for up to three or more days.

The designation “superabsorbent” refers here to a water-swellable, substantially water-insoluble material in fiber form which is capable of absorbing at least around 10 times, preferably around 20 times, and more preferably around 50 times or more of its weight of water. The superabsorbent material here may be formed of an organic material, which may comprise synthetic and/or natural material, such as, for example, agar, pectin and guar gum, or else synthetic materials, such as, for example, synthetic hydrogel polymers. Synthetic hydrogel polymers comprise, for example, carboxymethylcellulose, alkali metal salts of polyacrylic acids, alkali metal salts of polymethacrylic acids, polyacrylamides, polyvinyl alcohol, ethylene-maleic anhydride copolymers, polyvinyl ethers, hydroxypropylcellulose, polymers and copolymers of vinylsulfonic acid, polyacrylates, polymethacrylates, polyacrylate-polymethacrylate copolymers, polyacrylamides, and the like. Especially preferred as superabsorbent fibers are those fibers which are made of alkali metal salts of polyacrylic acids, alkali metal salts of polymethacrylic acids, polyacrylates, polymethacrylates or polyacrylate-polymethacrylate copolymers. The hydrogel polymers are preferably slightly crosslinked, in order to render the materials substantially insoluble in water. The crosslinking may be accomplished, for example, by irradiation or by the forming of covalent bonds, ionic bonds, van der Waals bonds or hydrogen bonds. Suitable materials are available from a variety of manufacturers, such as BASF and Stockhausen Inc.

Additionally provision is preferably made here for the first nonwoven layer to comprise, as absorbing material, a fibrous nonwoven which comprises a fiber mixture of at least 20 wt % of superabsorbent fibers, at least 10 wt % of viscose or cellulose fibers, and at least 5 wt % of thermoplastic fibers. With very particular preference the second absorbing layer comprises as absorbing material a fibrous nonwoven which comprises a fiber mixture of at least 40 wt % of superabsorbent fibers, at least 20 wt % of viscose or cellulose fibers, and at least 5 wt % of thermoplastic fibers. The fraction of supporting fibers in the fibrous nonwoven results in a uniform distribution, at the start of the absorption, of the volume of fluid to be absorbed.

The two layers, and also other layers, where they are connected by means of overstitching, are fed together to a warp knitting machine and are connected to one another by means of an elastic stitching thread, which may be selected preferably from a group composed of cotton spun crepe threads, cotton twisted crepe threads, textured polyamide yarns, textured polyester yarns, rubber threads or polyurethane elastane threads, or a combination thereof.

The stitching thread may alternatively also be referred to as warp thread. The thread here runs in the machine direction of the warp knitting machine, and not transverse to it.

The fully made-up, more particularly stitched, fabric of the compression bandage preferably exhibits optimized stretching, and with particular preference the maximum stretchability of the compression bandage, which corresponds to a mandated optimal stretchability, and a stretching of the compression bandage that goes beyond it may be limited by a stretching threshold. In this way the reliability of application may be significantly increased, since even for untrained users it is possible to stretch the compression bandage maximally up to the stretching threshold, at which point not only the maximum stretchability but also, at the same time, the optimal stretching and hence the optimal compressive pressure are achieved, with the compression bandage being applied in this maximally stretched state. There are variety of ways in which the stretching threshold may be adjusted. A thermal treatment (including thermosetting), for example, may allow the elasticity to be adjusted.

In order to achieve an optimal cushioning effect, where the bandage serves as a cushioning bandage, it may be preferable for the thickness of the nonwoven layer serving as cushioning layer to be 0.3-12 mm, preferably 0.4-6 mm and more preferably 0.5-3 mm, with particular preference 0.6-1.2 mm.

In the case of the preferred connection by means of a stitchbonding method, the two layers in the tension-released state, after the stitched-together fabric has been processed further by means of make-up in the longitudinal direction to form the compression bandage, are placed in waves such that an irregular surface of the compression bandage is formed. On the basis of this irregular surface, in addition to the primary function as an equalization layer of the compression bandage and the secondary function of the regulatable stretchability and hence increased reliability of application, a further function achieved is that, by virtue of the waves giving rise to a pattern on the surface composed of elevations of material and depressions of material that is not completely eliminated even in the maximally stretched state, and so in this way additionally a massage and/or drainage effect is developed during therapy.

In order to achieve a further improvement in reliability of application, provision may be made for a cohesive coating to be provided on the side facing away from the wearer, more particularly on the side of the compression bandage that faces away from the first nonwoven layer, more particularly on the second nonwoven layer.

One possible embodiment of the compression bandage is a bandage composed of two nonwoven plies of polyester material, with superabsorbent fibers in at least one of the two plies, the two-ply base textile having been obtained by overstitching a rigid polyester nonwoven fabric with permanently elastic elastane threads in the longitudinal direction, using the MALIWATT stitchbonding technique. The MALIWATT stitchbonding technique is described in Malimo Nähwirktechnologie, Ploch, Böttcher, Scharch, VEB Fachbuchverlag Leipzig, 1978, 1^{st} edition. This compression bandage is coated on one surface with a cohesively adhering bonding agent, in order to achieve the desired cohesive adhesion effect.

This bandage comprises the following composition over both layers:

65% polyester, 8% elastane, 7% synthetic rubber, basis weight stretched 105 +/−20 g/sqm according to DIN 61632, stretchability/retraction according to DIN 61632:65 +/−20%.

For application of a resting pressure, provision may be made, furthermore, for the compression bandage of the invention to be combined with, more particularly overwrapped, with a further compression bandage, which may take the form, for example, of a long-stretch bandage—that is, to provide, from the start of stretching, a compressive pressure which, however, does not increase abruptly.

The further bandage used for the resting pressure is preferably an elastic, cohesively adhering nonwoven bandage of type 752, tradename NOWOPRESS 752, manufacturer: Karl Otto Braun GmbH & Co, K G, Wolfstein, Germany, as a compression bandage having long-stretch properties, the base textile having been obtained by overstitching a rigid polypropylene nonwoven fabric with permanently elastic elastane threads in the longitudinal direction, using the MALIWATT stitchbonding technique. The MALIWATT stitchbonding technique is described in Malimo Nähwirktechnologie, Ploch, Böttcher, Scharch, VEB Fachbuchverlag Leipzig, 1978, 1^{st} edition. This bandage plie is coated on both surfaces with a cohesively adhering bonding agent based on polyisoprene rubber, in order to achieve the desired cohesive adhesion effect. The construction of the bandage here is as follows:

60% polypropylene, 12% elastane, 28% IR rubber
Base nonwoven: PP spunbonded nonwoven, 35 g/sqm, thermally embossed
Stitching thread: 133 dtex elastane (DORLASTAN, BAYER)

Stitching thread density: 45 threads per 10 cm width

Stitching thread stitch length: stitching 3 mm, open pillar stitch
Basis weight, stretched: 59 g/sqm
Elasticity in longitudinal direction (warp direction) Stretchability/retraction according to DIN 61632: 160%/99%
Adhesive force, side A/B: 60 cN/cm.

The invention accordingly also relates to a combination dressing composed of the compression bandage described, with first and second nonwoven layers, which provides in particular a working pressure for the compression therapy, and of a further bandage, configured in particular as a long-stretch bandage for the resting pressure.

Division into the categories of short-stretch, medium-stretch or long-stretch bandage is made according to stretchability and may be gathered for example from P. Asmussen, B. Söllner, Kompressionstherapie Prinzipien and Praxis [Compression therapy—principles and practice], Verlag Urban & Fischer in Elsevier, 2004, on page 121. The stretchabilities here are determined according to DIN 61632.

The invention is described in more detail below with reference to a drawing. Further advantages and features of the invention are additionally apparent from the rest of the application documents.

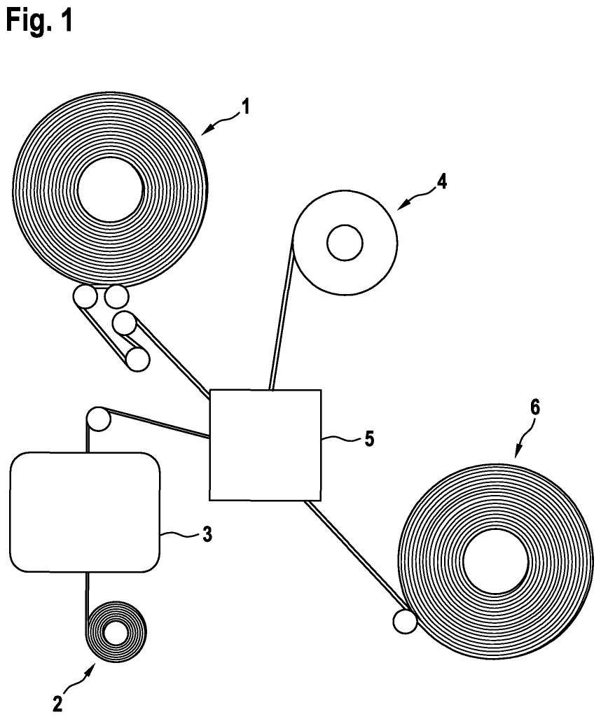

In the drawing, FIG. 1 shows a method for producing a compression bandage of this kind.

FIG. 1 depicts how a first nonwoven layer 2, provided with superabsorbent fibers, and a second nonwoven layer are each supplied as roll material. This first nonwoven layer 2 may consist of a nonwoven prefabricated with superabsorbent fibers, or may consist completely of superabsorbent fibers, or a nonwoven material may be covered with superabsorbent fibers in the facility 3. In the preferred case, the two nonwoven materials 1 and 2 are connected to one another in a stitchbonding method by means of a warp knitting machine, which carries the reference numeral 5; the elastifying agents 4, which in this case take the form of elastane threads and serve as warp threads, are supplied to the warp knitting machine 5. The stitchbonding method operates here with a hook, by means of which the layers are overstitched with the elastic stitching thread. The stitching thread is an elastically prestretched thread, which is introduced with a mandated stitch length and a mandated stitching thread tension, and which accordingly, after release of tension from the elastic material, results in a contraction of the plied assembly (fabric).

Other elastifying agents may also be used and supplied, alternatively.

If no stitchbonding method is used, a different facility 5 may be used, in which the plies are connected to one another and elastified.

The material connected mutually via the elastic stitching thread in particular is then wound up to form a roll 6; optionally, this may be preceded by make-up in the longitudinal direction to form bandages.

The compression bandages embodied in this way correspond in their characteristics to a short-stretch bandage, which initially exhibits approximately no increase in force on stretching, before then realizing an abrupt increase and hence a stretching threshold, with the stretching threshold here being situated within the range of the optimal therapeutic pressure, meaning that, on maximal stretching of the compression bandage, it is possible simultaneously to establish the optimal therapeutic pressure for compression therapy, when the bandage is applied with the aforementioned pre-tension.

By virtue of the superabsorbent fibers contained in the first nonwoven layer, wound exudates can be bound and can be led away from the wound. As a result, wound healing can be positively influenced, since in this way enzymes which can have an adverse effect on healing are removed from the wound.

1. A compression bandage comprising a first nonwoven layer and at least one second nonwoven layer, connected areally to the first nonwoven layer, wherein superabsorbent fibers are provided in the first nonwoven layer.

2. The compression bandage of claim 1, wherein in the applied state the first nonwoven layer is directed toward a wearer of the compression bandage.

3. The compression bandage of claim 1, wherein the first nonwoven layer is a thermofused nonwoven.

4. The compression bandage of claim 1, wherein the second nonwoven layer is a thermobonded nonwoven.

5. The compression bandage of claim 1, wherein the two nonwoven layers are connected to one another by means of stitchbonding methods via an elastic stitching thread.

6. The compression bandage of claim 1, wherein the material of one or both of the layers is nonelastic.

7. The compression bandage of claim 1, wherein the thickness of the first nonwoven layer is 0.6-1.2 mm.

8. The compression bandage of claim 1, wherein the nonwoven material of the first nonwoven layer and/or second nonwoven layer comprises polyester.

9. The compression bandage of claim 1, wherein the superabsorbent fibers consist of polyacrylate.

10. The compression bandage of claim 5, wherein the elastic stitching thread is selected from a group composed of cotton spun crepe threads, cotton twisted crepe threads, textured polyamide yams, textured polyester yams, rubber threads or polyurethane elastane threads, and combinations thereof.

11. The compression bandage of claim 1, wherein the compression bandage has a stretching threshold which indicates the maximum stretchability.

12. The compression bandage of claim 11, wherein the maximum stretchability corresponds to a therapeutically mandated application pressure of the compression bandage on a limb of a wearer.

13. The compression bandage of of claim 1, wherein a side of the compression bandage pointing away from the wearer has a cohesive coating.

14. A compression bandage combination comprising a first compression bandage of which is the compression bandage of claim 1, and also a second compression bandage applied over the first compression bandage.

15. The compression bandage combination of claim 14, wherein in the applied state, the first compression bandage exerts a working pressure and the second compression bandage a resting pressure on a limb.

16. The compression bandage of claim 2, wherein in the applied state the first nonwoven layer is directed toward a wearer of the compression bandage and is configured as a cushioning layer.

17. The compression bandage of claim 8, wherein the nonwoven material of the first nonwoven layer and/or second nonwoven layer consists of polyester.
